(19) United States
(12) Patent Application Publication
(10) Pub. No.: US 2026/0151602 A1
(43) Pub. Date: Jun. 4, 2026

(54) SYSTEMS AND METHODS FOR STENT PLACEMENT

(71) Applicant: BOSTON SCIENTIFIC SCIMED, INC., Maple Grove, MN (US)

(72) Inventors: SHANE O'KEEFFE, Tuam (IE); DAVID GILES, Tuam (IE); FIONN STAPLETON, Oranmore (IE); KEITH O'REILLY, Galway (IE); GARY GILMARTIN, Foxford (IE); DANIEL TUCK, Galway (IE); THOMAS MARTIN KEATING, Tuam (IE); JOHN THOMAS O'DRISCOLL, Gort (IE)

(73) Assignee: BOSTON SCIENTIFIC SCIMED, INC., Maple Grove, MN (US)

(21) Appl. No.: 19/407,980

(22) Filed: Dec. 3, 2025

Related U.S. Application Data
(60) Provisional application No. 63/727,924, filed on Dec. 4, 2024.

(51) Int. Cl. A61M 27/00; A61F 2/04; A61F 2/966

(52) U.S. Cl. CPC A61M 27/002; A61F 2/04; A61F 2/966; A61F 2002/041; A61F 2002/045; A61M 2210/1053

(57) ABSTRACT

Medical systems for positioning a stent along a tissue pathway are disclosed. A system includes an inner shaft having a lumen, a proximal end coupled to the handle and a distal end coupled to a tip member. The system also includes an outer shaft having a proximal end region and a lumen, wherein at least a portion of the inner shaft extends within the lumen of the outer shaft. The system also includes a stent positioned between an inner surface of the outer shaft and an outer surface of the inner shaft. Further, the tip member includes a first engagement member configured to engage and anchor the tip member to a tissue of the tissue pathway before the stent engages the tissue pathway.

### CROSS-REFERENCE TO RELATED APPLICATIONS

The application claims the benefit of U.S. Provisional Patent Application Ser. No. 63/727,924, filed on Dec. 4, 2024, the disclosure of which is incorporated herein by reference.

### TECHNICAL FIELD

This application relates generally to medical methods and devices. More specifically, the present disclosure relates to stents, stent delivery systems and methods for their use in establishing drainage pathways with medical procedures.

### BACKGROUND

A wide variety of medical devices have been developed for medical use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. A system for positioning a stent along a tissue pathway includes a handle. The system also includes an inner shaft having a lumen, a proximal end coupled to the handle and a distal end coupled to a tip member. The system also includes an outer shaft having a proximal end region and a lumen, wherein at least a portion of the inner shaft extends within the lumen of the outer shaft. The system also includes a stent positioned between an inner surface of the outer shaft and an outer surface of the inner shaft. Further, the tip member includes a first engagement member configured to engage and anchor the tip member to a tissue of the tissue pathway before the stent engages the tissue pathway.

Alternatively or additionally to any of the embodiments above, wherein the first engagement member includes a proximal end region and a distal end region, and wherein the proximal end region is coupled to the tip member, and wherein the distal end region is configured to engage and anchor the tip member to a tissue of the tissue pathway.

Alternatively or additionally to any of the embodiments above, wherein the first engagement member is configured to shift between a first constrained configuration and a second expanded configuration.

Alternatively or additionally to any of the embodiments above, wherein the first engagement member is biased to the second expanded configuration.

Alternatively or additionally to any of the embodiments above, wherein the outer shaft is configured to shift between a first position and a second position, and wherein the outer shaft is configured to extend over and maintain the first engagement member in the first constrained configuration when the outer shaft is in the first position.

Alternatively or additionally to any of the embodiments above, wherein shifting the outer shaft from the first position to the second position uncovers the first engagement member, and wherein the first engagement member is configured to shift from the first constrained configuration to the second expanded configuration as the outer shaft uncovers the first engagement member.

Alternatively or additionally to any of the embodiments above, wherein shifting the outer shaft from the second position to the first position covers the first engagement member, and wherein the first engagement member is configured to shift from the second expanded configuration to the first constrained configuration as the outer shaft covers the first engagement member.

Alternatively or additionally to any of the embodiments above, wherein the first engagement member disengages from the tissue of the tissue pathway as the outer shaft is shifted from the second position to the first position and covers the first engagement member.

Alternatively or additionally to any of the embodiments above, wherein the tip member includes a proximal region and a distal region, and wherein the first engagement member is positioned along the proximal region, and wherein the distal region includes a taper.

Alternatively or additionally to any of the embodiments above, wherein the tip member further includes a channel extending along a longitudinal axis of the proximal region, and wherein the engagement member is configured to nest within the channel when in the first constrained configuration.

Alternatively or additionally to any of the embodiments above, wherein the tip member further includes a second engagement member configured to engage and anchor the tip member to a tissue of the tissue pathway before the stent engages the tissue pathway.

Alternatively or additionally to any of the embodiments above, wherein the first engagement member is circumferentially spaced away from the second engagement member along a proximal region of the tip member.

Alternatively or additionally to any of the embodiments above, wherein the first engagement member includes a fin positioned along an outer surface of the tip member.

Alternatively or additionally to any of the embodiments above, wherein the first engagement member is configured to assume a curved configuration to engage and anchor the tip member to the tissue of the tissue pathway.

Another system for positioning a stent along a tissue pathway includes a handle. The system also includes an inner shaft having a lumen, a proximal end coupled to the handle and a distal end coupled to a tip member. The system also includes an outer shaft having a proximal end region and a lumen, wherein at least a portion of the inner shaft extends within the lumen of the outer shaft. The system also includes a grip member coupled to the proximal end region of the outer shaft, a stent positioned between an inner surface of the outer shaft and an outer surface of the inner shaft and a plurality of engagement members coupled to the tip member. Further, the plurality of engagement members are configured to engage and anchor the tip member to a tissue of the tissue pathway before the stent engages the tissue pathway.

Alternatively or additionally to any of the embodiments above, wherein each of the plurality of engagement members includes a proximal end region and a distal end region, and wherein the proximal end region of each of the plurality of engagement members is coupled to the tip member, and wherein the distal end region of each of the plurality of engagement members is configured to engage and anchor the tip member to a tissue of the tissue pathway.

Alternatively or additionally to any of the embodiments above, wherein the plurality of engagement members are configured to shift between a first constrained configuration and a second expanded configuration.

Alternatively or additionally to any of the embodiments above, wherein the outer shaft is configured to shift between a first position and a second position, and wherein the outer shaft is configured to extend over and maintain the plurality of engagement members in the first constrained configuration when the outer shaft is in the first position.

Alternatively or additionally to any of the embodiments above, wherein shifting the outer shaft from the first position to the second position uncovers the plurality of engagement members, and wherein plurality of first engagement members are configured to shift from the first constrained configuration to the second expanded configuration as the outer shaft uncovers the plurality of engagement members.

A method for positioning a drainage stent between a biliary duct and the stomach includes positioning a distal end region of a stent delivery system within the biliary duct and a proximal end region of the stent delivery system within the stomach, the stent delivery system including a handle, an inner shaft having a lumen, a proximal end coupled to the handle and a distal end coupled to a tip member. The delivery system also includes an outer shaft having a proximal end region and a lumen, wherein at least a portion of the inner shaft extends within the lumen of the outer shaft. The delivery system also includes a stent positioned between an inner surface of the outer shaft and an outer surface of the inner shaft and a plurality of engagement members coupled to the tip member. The method also includes retracting the outer shaft in a proximal direction, engaging the plurality of engagement members to a tissue of the biliary duct, engaging a distal end of the stent to a tissue of the biliary duct, engaging a proximal end of the stent to a tissue of the stomach, advancing the outer shaft in a distal direction and disengaging the plurality of engagement members to a tissue of the biliary duct.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example stent delivery system for accessing the stomach and/or biliary tract.

FIG. 2 is a perspective view of a portion of the system of FIG. 1 in a first configuration.

FIG. 3 is a perspective view of a portion of the system of FIG. 1 in a second configuration.

FIG. 4 is a perspective view of a portion of another example stent delivery system.

FIG. 5 is a perspective view of a portion of another example stent delivery system.

FIG. 6 is a perspective view of a portion of another example stent delivery system.

FIG. 7 is a perspective view of a portion of another example stent delivery system.

FIG. 8 is a partial cross-sectional view of a portion of the stent delivery system of FIG. 1 in a first configuration.

FIG. 9 is a partial cross-sectional view of a portion of the stent delivery system of FIG. 1 in a second configuration.

FIGS. 10-15 illustrate a method for deploying a stent within a portion of the digestive system.

FIG. 16 is a perspective view of a portion of another embodiment of a stent delivery system.

FIG. 17 is a front view of the portion of the stent delivery system shown in FIG. 16.

FIG. 18 illustrates a portion of another example stent delivery system in a first configuration.

FIG. 19 illustrates a portion of the example stent delivery system of FIG. 18 in a second configuration.

FIG. 20A illustrates a portion of another example stent delivery system.

FIG. 20B illustrates a portion of another example stent delivery system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Biliary obstruction may commonly be caused by a variety of conditions such as pancreatic cancer, cholangiocarcinoma, or metastatic tumors compressing the bile ducts. Different approaches may be utilized for managing biliary obstruction, such as endoscopic retrograde cholangiopancreatography or percutaneous transhepatic biliary drainage. However, when such approaches are not feasible or have failed, alternative medical procedures may be utilized. For example, endoscopic ultrasound-guided hepaticogastrostomy (EUS-HGS) is a minimally invasive procedure that establishes a drainage pathway between a duct (e.g., biliary duct, hepatic duct, bile duct, etc.) and the stomach to bypass a biliary obstruction.

An endoscopic ultrasound-guided hepaticogastrostomy procedure may require precise placement of a drainage stent between an obstructed duct and the stomach. The procedure may include passing an endoscope into the stomach through a first organ or structure, such as the esophagus. Once the endoscope is positioned in the stomach, a stent delivery system may be passed through a working channel of the endoscope, whereby the stent delivery system may be utilized to create a fistula between the stomach and the obstructed duct, thereby forming a pathway for placement of a stent (e.g., a covered stent) configured to drain fluid from the obstructed duct into the stomach. The fluid may then exit the body via the large and small intestines.

When deploying a drainage stent between adjacent body lumens, organs, or other structures, it is typically necessary to penetrate both a wall of the first body organ (e.g., stomach) through which access is established and a wall of a second body lumen (e.g., biliary duct, hepatic duct, bile duct, etc.). When initially forming such access penetrations, there may be a risk of leakage from either or both of the access body organ and the target body lumen into the surrounding space. In some procedures, such as those involving endoscopic ultrasound-guided hepaticogastrostomy, loss of body fluid into surrounding tissues and body cavities can present a substantial risk to the patient.

Further, the precise placement of a covered drainage stent within the target body lumen (e.g., biliary duct, hepatic duct, bile duct, etc.) may minimize the leakage of body fluid into surrounding tissues and body cavities of the patient. Accordingly, it can be appreciated that minimizing movement of the stent delivery system and or the endoscope may improve the precision of the placement of a covered drainage stent deployed within the a target body lumen (e.g., biliary duct, hepatic duct, bile duct, etc.) of a patient. For example, stent delivery systems including a tip member (e.g., nosecone) that may temporarily engage a wall of a target body lumen (e.g., biliary duct, hepatic duct, bile duct, etc.) may minimize the movement of one or more components of the stent delivery system relative to the body lumen while the stent is deployed from the stent delivery system. Stent delivery systems including a tip member configured to temporarily engage the wall of the body lumen while a stent is deployed from the stent delivery system are disclosed herein.

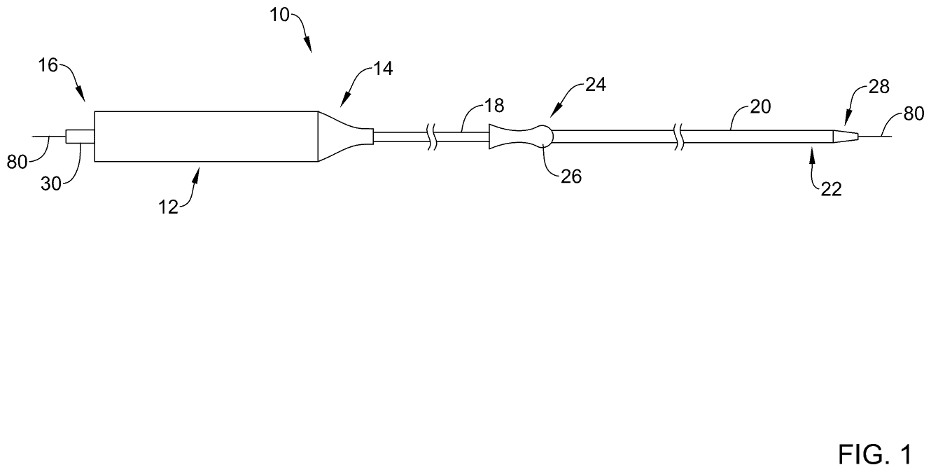

FIG. 1 illustrates an example system 10 (e.g., stent delivery system) for establishing a drainage pathway and deploying a drainage stent between a first body organ (e.g., stomach) and a body lumen or cavity (e.g., biliary duct, hepatic duct, bile duct, etc.). In at least some instances, the system 10 is configured to be used with an endoscope (e.g., and/or with an endoscopic ultrasound device) for performing an endoscopic ultrasound-guided hepaticogastrostomy or other similar medical procedure. As will be described in more detail herein, the system 10 may be advanced through at least a portion of the digestive tract (e.g., esophagus) of a patient, passed through a wall of the stomach and into a lumen/duct along the biliary tract, whereby a covered drainage stent may then be deployed to connect the biliary duct (or other duct) to the stomach cavity.

The system 10 may include a handle or handle assembly 12. The handle 12 may include a distal end region 14 and a proximal end region 16. As illustrated in FIG. 1, the distal end region 14 may be coupled to the proximal end region of an inner shaft 18. Further, the inner shaft 18 may extend through a lumen of an outer shaft 20.

Additionally, FIG. 1 illustrates that the distal end of the inner shaft 18 may be attached to a tip member 28. In some examples, the tip member 28 may include a marker configured to help a user guide the tip member 28 during a medical procedure. It can be appreciated that the marker positioned on the tip member 28 may be formed from a band or ring of solid radiopaque metal, such as gold, tungsten, tantalum, platinum other similar materials.

FIG. 1 further illustrates that the outer shaft 20 may include a distal end region 22 and a proximal end region 24. FIG. 1 illustrates that, in some examples, a distal end portion of the tip member 28 may be positioned adjacent to the distal end of the outer shaft 20. In other words, the tip member 28 may include a proximal face which contacts and/or abuts a distal face of the outer shaft 20.

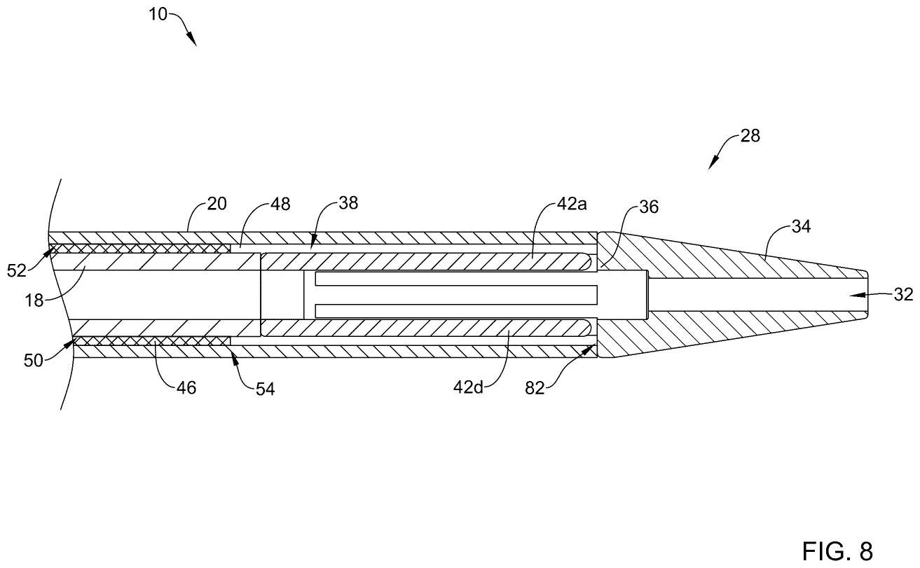

FIG. 1 further illustrates that the proximal end region 24 of the outer shaft 20 may include grip member 26. The grip member 26 may be configured to include an ergonomic shape which permits a user to easily and comfortably grasp the grip member 26. As will be discussed in greater detail below, the grip member 26 may permit a user to actuate the outer shaft 20 relative to the inner shaft 18 and the handle 12. For example, the system 10 may be configured to permit a user to grasp the handle 12 with one hand while using their opposite hand to grasp and pull the grip member 26 toward the handle 12, thereby translating (e.g., sliding) the outer shaft 20 relative to the inner shaft 18. As will be discussed in greater detail herein, the distal-to-proximal translation of the outer shaft 20 relative to the inner shaft 18 may deploy a stent 46 (shown in FIG. 8) positioned along a stent holding region between the outer shaft 20 and the inner shaft 18.

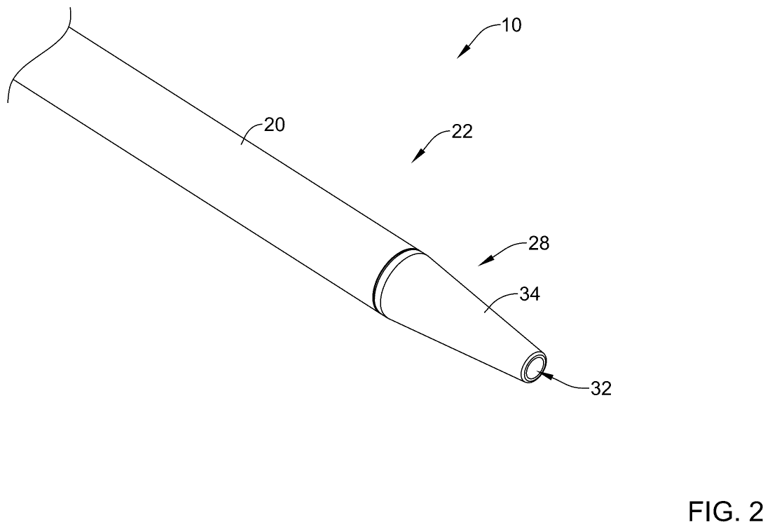

FIG. 1 further illustrates that the proximal end region 16 of the handle 12 may include a port 30 (e.g., luer fitting, etc.). The port 30 may be configured to permit the attachment of a syringe, Y-adaptor, etc. thereto. It can be appreciated that the port 30 may permit the injection of fluid (e.g., contrast, saline, etc.) into a lumen of the inner shaft 18. Further, it can be appreciated that port 30 may permit aspiration of fluid through the lumen of the inner shaft 18. Further yet, it can be appreciated that the port 30 may permit a guidewire 80 to be inserted and extend within the lumen 32 (shown in FIG. 2) of the inner shaft 18.

FIG. 2 illustrates a perspective view of the distal end region of the system 10 shown in FIG. 1. FIG. 2 illustrates a distal end region 22 of the outer shaft 20 positioned adjacent to the tip member 28. It can be appreciated from FIG. 2 that the distal end of the outer shaft 20 may engage (e.g., abut, contact, etc.) a proximal facing surface of the tip member 28. FIG. 2 further illustrates that the tip member 28 may include distal tapered portion 34.

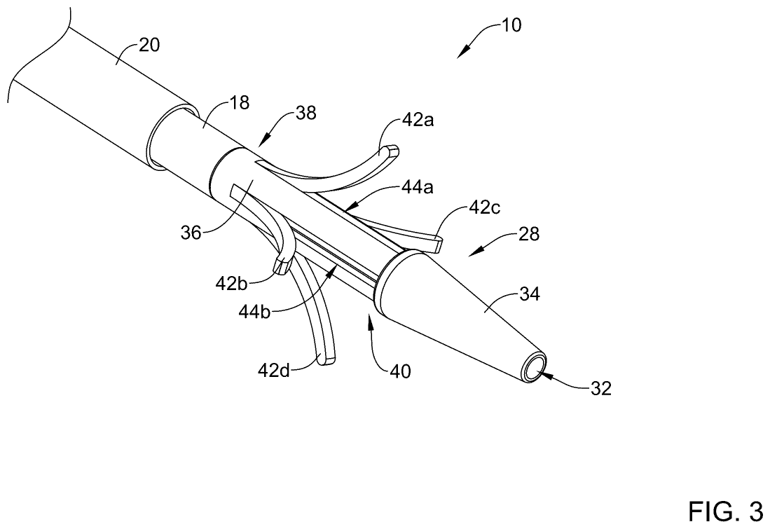

FIG. 3 illustrates a perspective view of the system 10 shown in FIG. 2, whereby the outer shaft 20 has been retracted in a distal-to-proximal direction relative to the inner shaft 18 and the tip member 28. It can be appreciated that the distal-to-proximal retraction of the outer shaft 20 relative to the inner shaft 18 and the tip member 28 may be accomplished via a user grasping the handle 12 with one hand while using their opposite hand to grasp and pull the grip member 26 (shown in FIG. 1) toward the handle 12, thereby translating the outer shaft 20 relative to the inner shaft 18.

FIG. 3 further illustrates that the tip member 28 may include a proximal portion 36 extending proximally from the distal tapered portion 34. The proximal portion 36 of the tip member 28 may include a proximal end region 38 and a distal end region 40. FIG. 3 illustrates that a distal end of the inner member 18 may be attached to the proximal end region 38 of the tip member 28. It can be appreciated that a lumen of the inner member 18 may be in fluid communication with the lumen 32 of the tip member 28. Accordingly, it can be appreciated that a guidewire 80 (shown in FIG. 1) may be advanced through the lumen of the inner member 18 and the lumen 32 of the tip member 28.

FIG. 3 further illustrates that the tip member 28 may include one or more engagement members 42a, 42b, 42c, 42d (e.g., prongs, tines, etc.) extending along the proximal portion 36 of the tip member 28. It can be appreciated from FIG. 3 that each of the engagement members 42a, 42b, 42c, 42d may include a first end which is attached to the proximal end region 38 of the proximal portion 36. It can be further appreciated from FIG. 3 that each of the engagement members 42a, 42b, 42c, 42d may include a second end, opposite the first end, which is unattached to the proximal portion 36.

FIG. 3 further illustrates that each of the engagement members 42a, 42b, 42c, 42d may be configured to shift from a first configuration in which the engagement members 42a, 42b, 42c, 42d are nested within a channel (e.g., groove, slot, etc.) extending along the proximal portion 36 of the tip member 28 to a second configuration in which the engagement members 42a, 42b, 42c, 42d are expanded (e.g., flexed) radially outward from the proximal portion 36 of the tip member 28. For example, FIG. 3 illustrates that the engagement members 42a, 42b may be configured to nest within channels 44a, 44b, respectively, which extend along the proximal portion 36 of the tip member 28. It can be appreciated that the engagement members 42c, 42d may be configured to nest within channels similar to the channels 44a, 44b, however, the channels within which the engagement members 42c, 42d are configured to nest are hidden from view in FIG. 3.

Additionally, it can be appreciated that each of the engagement members 42a, 42b, 42c, 42d may be biased to assume the radially expanded (e.g., flexed) configuration illustrated in FIG. 3 when they are not constrained by (e.g., positioned underneath) the outer shaft 20. However, as discussed herein, when positioned underneath the outer shaft 20 (as illustrated in FIG. 2), each of the engagement members 42a, 42b, 42c, 42d may be held in place by the outer and nested within its respective channel (e.g., engagement member 42a may nest within channel 44a, engagement member 42b may nest within channel 44b, etc.). Further, retraction of the outer shaft 20 in a distal-to-proximal direction may permit each of the engagement members 42a, 42b, 42c, 42d to expand (e.g., flex) radially outward away from the tip member 28 to the configuration illustrated in FIG. 3. It can be further appreciated that the proximal-to-distal advancement of the outer shaft 20 relative to the tip member 28 may re-position (e.g., re-nest) each of the engagement members 42a, 42b, 42c, 42d into their respective channels as the outer shaft 20 is advanced over top of each of the engagement members 42a, 42b, 42c, 42d.

As discussed herein, it can be appreciated that each of the four engagement members 42a, 42b, 42c, 42d may be configured to temporarily engage a wall of a target body lumen (e.g., biliary duct, hepatic duct, bile duct, etc.) while a stent (such as stent 46 shown in FIG. 8) is deployed from the stent delivery system 10. Engagement of the engagement members 42a, 42b, 42c, 42d with a wall of a target body lumen (e.g., biliary duct, hepatic duct, bile duct, etc.) while a stent is deployed may minimize the movement of the stent delivery system 10 and/or the endoscope during deployment, thereby improving the precision of the placement of a covered drainage stent within the a target body lumen (e.g., biliary duct, hepatic duct, bile duct, etc.) of a patient.

FIG. 3 illustrates that the tip member 28 includes four engagement members 42a, 42 b, 42 c, 42 d. However, it can be appreciated that the tip member 28 may include 1, 2, 3, 4, 5, 6, 7, 8 or more engagement members. Further, FIG. 3 illustrates the engagement members 42a, 42b, 42c, 42d spaced substantially equidistant from one another around the circumference of the proximal portion 36. However, in other examples the engagement members 42a, 42b, 42c, 42d may be spaced substantially unequally around the circumference of the proximal portion 36.

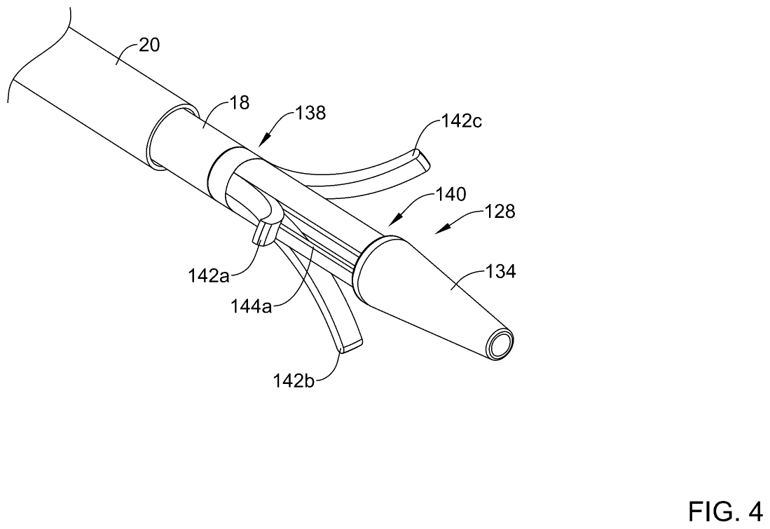

FIGS. 4-7 illustrate example tip members which may be similar in form and function to the tip member 28 described herein. These examples are in addition to the example shape of the tip member 28 shown in FIGS. 1-3. The example shapes of the tip members shown in FIGS. 4-7 are schematic representations that depict alternative arrangements. The shapes shown in FIGS. 4-7 are intended as variations of the shape of the tip member 28 shown in FIG. 1, and can be utilized in the system 10 disclosed as alternatives of the tip member 28.

FIG. 4 illustrates another example tip member 128 which may be similar in form and function to the tip member 28 described herein. FIG. 4 illustrates that the tip member 128 may include a proximal portion 136 extending distally from a tapered portion 134. The proximal portion 136 may include a proximal end region 138 and a distal end region 140. The proximal end region 138 may be attached to a distal end of the inner shaft 18. FIG. 4 illustrates the inner shaft 18 extending within a lumen of the outer shaft 20.

FIG. 4 further illustrates that the tip member 128 may include three engagement members 142a, 142b, 142c which may be similar in form and function to the engagement members 42a, 42b, 42c, 42d described herein. For example, FIG. 4 illustrates that each of the engagement members 142a, 142b, 142c may be configured to assume the radially expanded (e.g., flexed) configuration illustrated in FIG. 4 when they are not constrained by the outer shaft 20. Like that described with respect to the engagement members 42a, 42b, 42c, 42d, it can be appreciated that when positioned underneath the outer shaft 20 (as illustrated in FIG. 2) each of the engagement members 142a, 142b, 142c may be configured to nest within its respective channel (e.g., engagement member 142a may nest within channel 144a, etc.). Further, retraction of the outer shaft 20 in a distal-to-proximal direction may permit each of the engagement members 142a, 142b, 142c to expand (e.g., flex) radially outward away from an outer surface of the tip member 128 to the configuration illustrated in FIG. 4. It can be further appreciated that the proximal-to-distal advancement of the outer shaft 20 relative to the tip member 128 may re-position each of the engagement members 142a, 142b, 142c into their respective channels as the outer is advanced over top of each of the engagement members 142a, 142b, 142c.

As discussed herein, it can be appreciated that each of the engagement members 142a, 142b, 142c may be configured to temporarily engage a wall of a target body lumen (e.g., biliary duct, hepatic duct, bile duct, etc.) while a stent (such as stent 46 shown in FIG. 8) is deployed from the stent delivery system 10. Engagement of the engagement members 142a, 142b, 142c with a wall of a target body lumen (e.g., biliary duct, hepatic duct, bile duct, etc.) while a stent is deployed may minimize the movement of the stent delivery system 10 and/or the endoscope during deployment, thereby improving the precision of the placement of a covered drainage stent within the a target body lumen (e.g., biliary duct, hepatic duct, bile duct, etc.) of a patient.

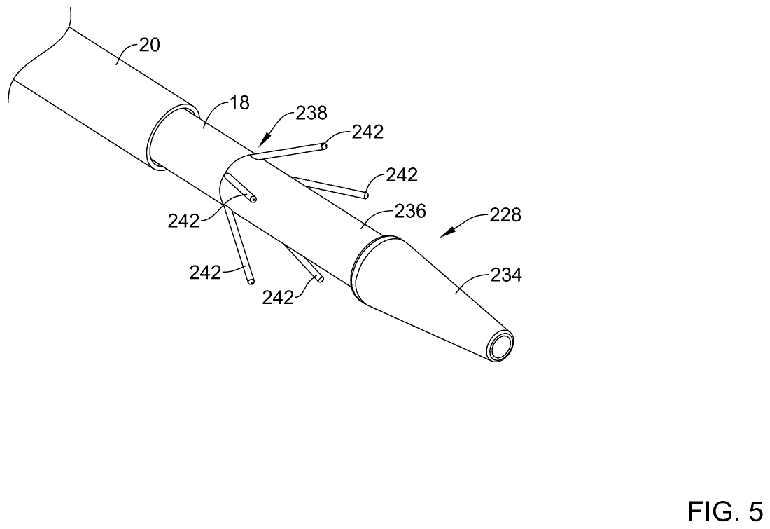

FIG. 5 illustrates another example tip member 228 which may be similar in form and function to the tip member 28 described herein. FIG. 5 illustrates that the tip member 228 may include a proximal portion 236 extending distally from a tapered portion 234. The proximal portion 236 may include a proximal end region 238. The proximal end region 238 may be attached to a distal end of the inner shaft 18. FIG. 5 illustrates the inner shaft 18 extending within a lumen of the outer shaft 20.

FIG. 5 further illustrates that the tip member 228 may include engagement members 242 which may be attached to the proximal end region 238. In some examples, the engagement members 242 may be shaped similarly to a cylindrical rod or shaft. It can be appreciated that each of the engagement members 242 may be configured to assume the radially expanded (e.g., flexed) configuration illustrated in FIG. 5 when they are not constrained by the outer shaft 20. It can be appreciated that when positioned underneath the outer shaft 20 (as illustrated in FIG. 2) each of the engagement members 242 may be configured to be positioned along the outer surface of the proximal portion 236 of the tip member 228. Further, retraction of the outer shaft 20 in a distal-to-proximal direction may permit each of the engagement members 242 to expand (e.g., flex) radially outward away from an outer surface of the proximal portion 236 to the configuration illustrated in FIG. 5. It can be further appreciated that the proximal-to-distal advancement of the outer shaft 20 relative to the tip member 228 may re-position each of the engagement members 242 along the outer surface of the proximal portion 236 as the outer shaft 20 is advanced over top of each of the engagement members 242.

As discussed herein, it can be appreciated that each of the engagement members 242 may be configured to temporarily engage a wall of a target body lumen (e.g., biliary duct, hepatic duct, bile duct, etc.) while a stent (such as stent 46 shown in FIG. 8) is deployed from the stent delivery system 10. Engagement of the engagement members 242 with a wall of a target body lumen (e.g., biliary duct, hepatic duct, bile duct, etc.) while a stent is deployed may minimize the movement of the stent delivery system 10 and/or the endoscope during deployment, thereby improving the precision of the placement of a covered drainage stent within the a target body lumen (e.g., biliary duct, hepatic duct, bile duct, etc.) of a patient.

FIG. 5 illustrates that the tip member 228 includes five engagement members 242. However, it can be appreciated that the tip member 228 may include 1, 2, 3, 4, 5, 6, 7,8 or more engagement members 242. Further, FIG. 5 illustrates the engagement members 242 spaced substantially equidistant from one another around the circumference of the proximal portion 236. However, in other examples the engagement members 242 may be spaced substantially unequally around the circumference of the proximal portion 236.

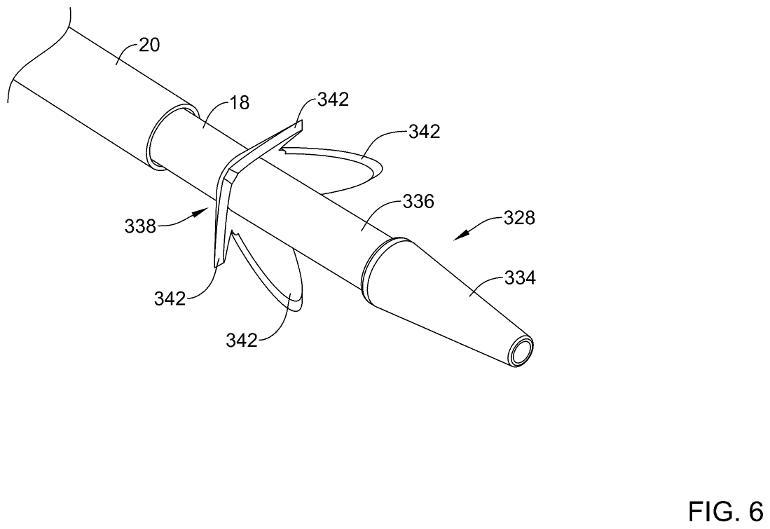

FIG. 6 illustrates another example tip member 328 which may be similar in form and function to the tip member 28 described herein. FIG. 6 illustrates that the tip member 328 may include a proximal portion 336 extending distally from a tapered portion 334. The proximal portion 336 may include a proximal end region 338. The proximal end region 338 may be attached to a distal end of the inner shaft 18. FIG. 6 illustrates the inner shaft 18 extending within a lumen of the outer shaft 20.

FIG. 6 further illustrates that the tip member 328 may include engagement members 342 which may be attached to the proximal end region 338. In some examples, the engagement members 342 may be substantially petal-shaped. It can be appreciated that each of the engagement members 342 may be configured to assume the radially expanded (e.g., flexed) configuration illustrated in FIG. 6 when they are not constrained by the outer shaft 20. It can be appreciated that when positioned underneath the outer shaft 20 (as illustrated in FIG. 2) each of the engagement members 342 may be configured to be positioned along the outer surface of the proximal portion 336 of the tip member 328. Further, retraction of the outer shaft 20 in a distal-to-proximal direction may permit each of the engagement members 342 to expand (e.g., flex) radially outward away from an outer surface of the proximal portion 336 to the configuration illustrated in FIG. 6. It can be further appreciated that the proximal-to-distal advancement of the outer shaft 20 relative to the tip member 328 may re-position each of the engagement members 342 along the outer surface of the proximal portion 336 as the outer shaft 20 is advanced over top of each of the engagement members 342.

As discussed herein, it can be appreciated that each of the engagement members 342 may be configured to temporarily engage a wall of a target body lumen (e.g., biliary duct, hepatic duct, bile duct, etc.) while a stent (such as stent 46 shown in FIG. 8) is deployed from the stent delivery system 10. Engagement of the engagement members 342 with a wall of a target body lumen (e.g., biliary duct, hepatic duct, bile duct, etc.) while a stent is deployed may minimize the movement of the stent delivery system 10 and/or the endoscope during deployment, thereby improving the precision of the placement of a covered drainage stent within the a target body lumen (e.g., biliary duct, hepatic duct, bile duct, etc.) of a patient.

FIG. 6 illustrates that the tip member 328 includes four engagement members 342. However, it can be appreciated that the tip member 328 may include 1, 2, 3, 4, 5, 6, 7, 8 or more engagement members 342. Further, FIG. 6 illustrates the engagement members 342 spaced substantially equidistant from one another around the circumference of the proximal portion 336. However, in other examples the engagement members 342 may be spaced substantially unequally around the circumference of the proximal portion 336.

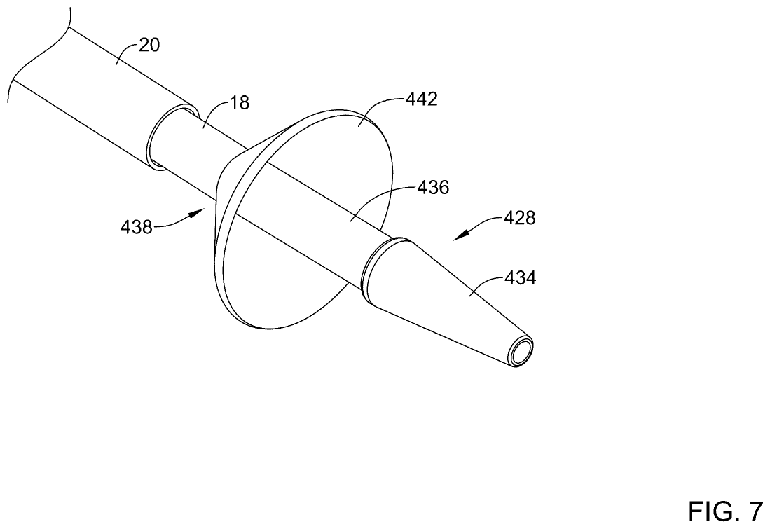

FIG. 7 illustrates another example tip member 428 which may be similar in form and function to the tip member 28 described herein. FIG. 7 illustrates that the tip member 428 may include a proximal portion 436 extending distally from a tapered portion 434. The proximal portion 436 may include a proximal end region 438. The proximal end region 438 may be attached to a distal end of the inner shaft 18. FIG. 7 illustrates the inner shaft 18 extending within a lumen of the outer shaft 20.

FIG. 7 further illustrates that the tip member 428 may include an engagement member 442 which may be attached to the proximal end region 438. It can be appreciated from FIG. 7 that the engagement member 442 may be substantially cone-shaped, whereby the engagement member 442 extends continuously around the circumference of the proximal portion 436. It can be appreciated that the engagement member 442 may be configured to assume the radially expanded (e.g., flexed) configuration illustrated in FIG. 7 when it is not constrained by the outer shaft 20. It can be appreciated that when positioned underneath the outer shaft 20 (as illustrated in FIG. 2) the engagement member 442 may be configured to be positioned along the outer surface of the proximal portion 436 of the tip member 428. Further, retraction of the outer shaft 20 in a distal-to-proximal direction may permit the engagement member 442 to expand (e.g., flex) radially outward away from an outer surface of the proximal portion 436 to the configuration illustrated in FIG. 7. It can be further appreciated that the proximal-to-distal advancement of the outer shaft 20 relative to the tip member 428 may re-position the engagement member 442 along the outer surface of the proximal portion 436 as the outer shaft 20 is advanced over top of the engagement member 442.

As discussed herein, it can be appreciated that the engagement members 442 may be configured to temporarily engage a wall of a target body lumen (e.g., biliary duct, hepatic duct, bile duct, etc.) while a stent (such as stent 46 shown in FIG. 8) is deployed from the stent delivery system 10. Engagement of the engagement member 442 with a wall of a target body lumen (e.g., biliary duct, hepatic duct, bile duct, etc.) while a stent is deployed may minimize the movement of the stent delivery system 10 and/or the endoscope during deployment, thereby improving the precision of the placement of a covered drainage stent within the a target body lumen (e.g., biliary duct, hepatic duct, bile duct, etc.) of a patient.

FIG. 8 illustrates a distal portion of the system 10 of FIG. 1. FIG. 8 illustrates the distal end of the outer shaft 20 positioned adjacent to (e.g., abutting, contacting, etc.) a proximal-facing surface 82 of the tip member 28. Further, FIG. 8 illustrates the inner shaft 18 extending within the lumen of the outer shaft 20 whereby the distal end region of the inner shaft 18 is coupled to a proximal end of the proximal portion 36 of the tip member 28. FIG. 8 further illustrates the outer shaft 20 extending over top of the proximal portion 36, including the engagement members 42a, 42b (it can be appreciated that the engagement members 42b, 42c are hidden from view in FIG. 8). FIG. 8 further illustrates that, when positioned over top of the engagement members 42a, 42b, the outer shaft 20 may position and maintain the engagement members 42a, 42d within their respective channels 44a (shown in FIGS. 3), 44d, as described herein with respect to FIGS. 2-3.

FIG. 8 further illustrates that the system 10 may further include a stent 46 positioned in a gap 48 (e.g., stent hold region) formed between the outer surface 50 of the inner shaft 18 and the inner surface 52 of the outer shaft 20. The stent 46 may be a self-expandable stent configured to expand from a first constrained configuration to a second expanded configuration. In some examples, the system 10 may be configured to deploy stents that are about 6 mm to about 110 mm long, or about 20 mm to about 100 mm long, or about 30 mm to about 90 mm long, or about 40 mm to about 80 mm long, or about 40 mm to about 70 mm long, or about 50 mm to about 100 mm long.

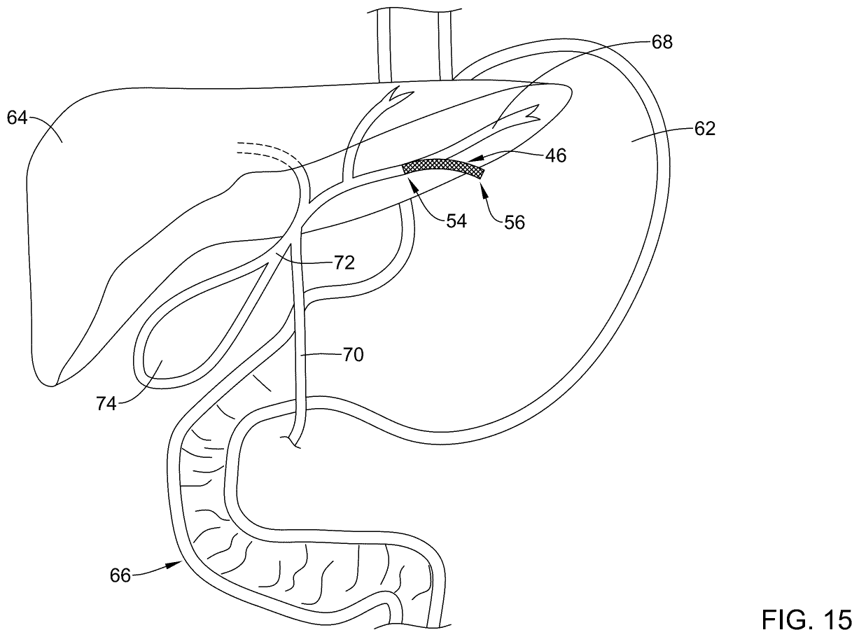

In some examples, the stent 46 may include a covering and/or coating positioned along a portion or the entire length of the stent 46. Further, in some examples, the distal end region 54, the proximal end region 56 (shown in FIG. 15) or both the distal and proximal end regions 54, 56 of the stent 46 may include a flared portion. It can be appreciated that the system 10 shown in FIG. 8 shows the stent 46 positioned in a pre-deployed configuration in which the outer shaft 20 extends over the entire length of the stent 46, thereby maintaining the stent 46 in a constrained, pre-deployed configuration.

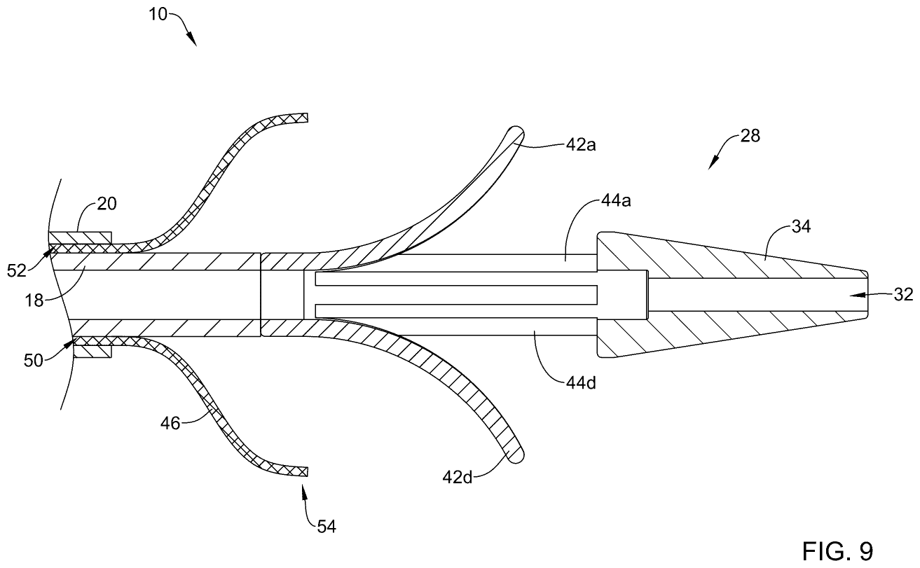

FIG. 9 illustrates that the distal-to-proximal retraction of the outer shaft 20 relative to the inner shaft 18, the stent 46 and the tip member 28. As described herein, distal-to-proximal retraction of the outer shaft 20 relative to the inner shaft 18 and the stent 46 may be accomplished via a user grasping the handle 12 with one hand while using their opposite hand to grasp and pull the grip member 26 (shown in FIG. 1) toward the handle 12, thereby translating the outer shaft 20 relative to the inner shaft 18.

FIG. 9 further illustrates that the distal-to-proximal retraction of the outer shaft 20 relative to the inner shaft 18 may sequentially uncover (e.g., expose) the engagement members 42a, 42d followed by the stent 46. For example, FIG. 9 illustrates that the distal-to-proximal retraction of the outer shaft 20 relative to the inner shaft 18 initially uncovers the engagement members 42a, 42d, thereby permitting the engagement members 42a, 42d to shift from a constrained configuration in which the engagement members 42a, 42d are nested with the channels 44a, 44b to the radially expanded configuration as illustrated in FIG. 3. FIG. 9 further illustrates that, after uncovering the engagement members 42a, 42d, the distal-to-proximal retraction of the outer shaft 20 relative to the inner shaft 18 further exposes the stent 46, thereby permitting the stent 46 to shift from a constrained configuration to an expanded configuration. It can be appreciated that the distal-to-proximal retraction of the outer shaft 20 may progressively uncover the stent 46 from distal end region 54 to the proximal end region 56 (shown in FIG. 15).

As will be discussed herein, it can be appreciated that the sequential uncovering of the engagement members 42a, 42d before the uncovering of the stent 46 may permit the tip member 28 to engage the a wall of a body vessel prior to the stent 46 being deployed. The engagement of the tip member 28 to the wall of a body vessel prior to the deployment of the stent 46 may stabilize the stent delivery system 10 during deployment of the stent 46, thereby permitting more precise deployment of the stent 46. While FIGS. 8-9 illustrate the radial expansion of the engagement members 42a, 42d, it can be appreciated that the engagement members 42b, 42c may also be radially expanded in a manner similar to engagement members 42a, 42d but are hidden from view in FIGS. 8-9

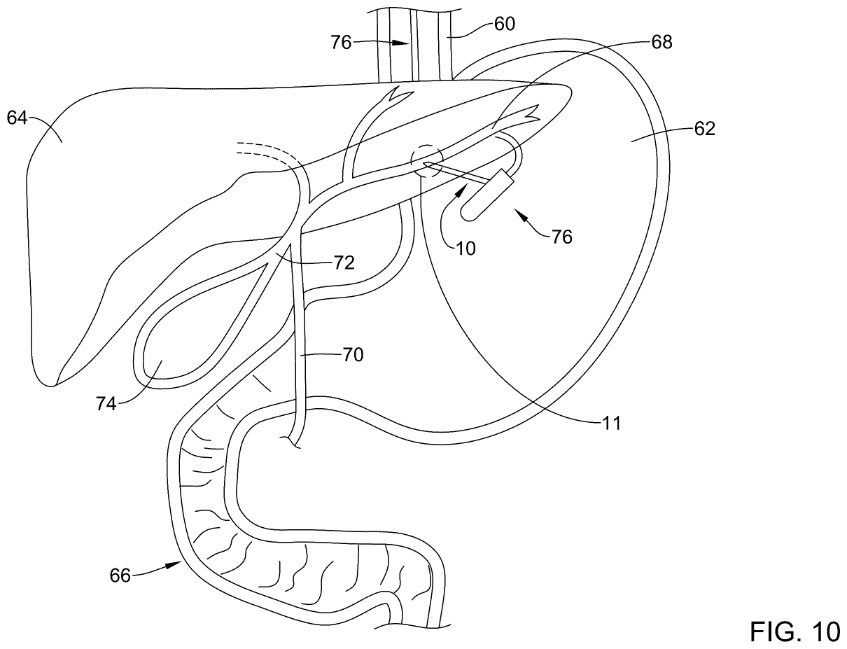

FIG. 10 illustrates an overview of the biliary system or tree. FIG. 10 illustrates the esophagus 60 connected to the stomach 62 (note that a portion of the liver 64 is masking the opening in which the esophagus 60 empties into the gastric lumen of the stomach 62). A portion of the duodenum 66 is shown extending from a portion of the stomach 62. Further, the biliary ducts, denoted by the reference numeral 68, are connected to the liver 64 and empty into the bile duct 70. Similarly, the cystic duct 72, being connected to the gall bladder 74, also empties into the bile duct 70.

As discussed herein, an endoscopic or biliary procedure may include advancing a medical device through the esophagus 60 to a suitable location within the stomach 62, whereby the medical device may be utilized to access a suitable location along the biliary tree and then performing the appropriate intervention. For example, FIG. 10 illustrates an endoscope 76 extending through the esophagus 60 to a suitable location within the stomach 62. The disclosure is not intended to be limited to disposing the endoscope 76 in the stomach 62 as other locations are contemplated including other portions of the digestive system including the stomach 62. For example, the system 10 and methods utilizing the system 10 may include draining the biliary tree between the bile duct 70 and the duodenum 66.

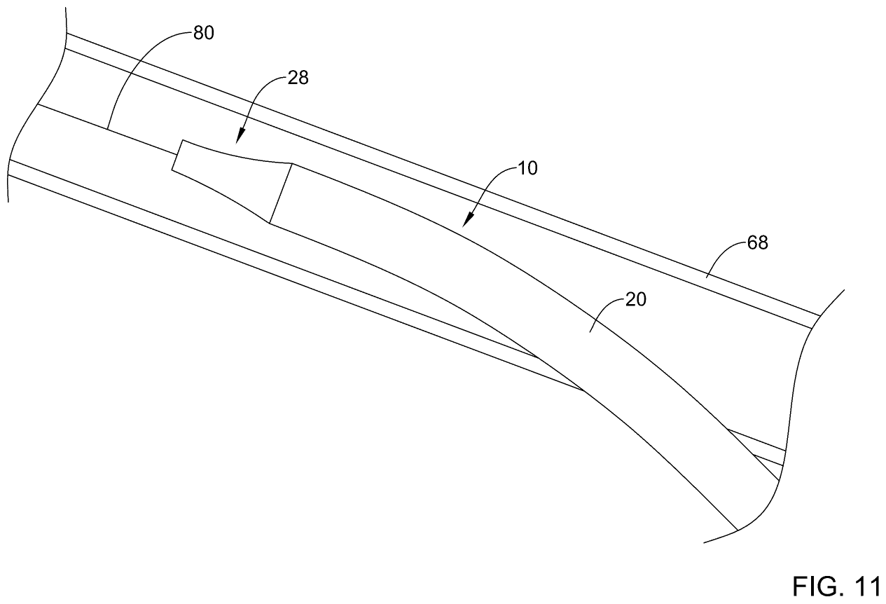

FIGS. 11-15 illustrate the deployment of the stent 40 within an example biliary duct 68. It can be appreciated that the initiation of the deployment of the stent 46 may occur at a time point soon after a fistula is created between the gastric lumen of the stomach 62 and the lumen of the biliary duct 68. Initiating the deployment of the stent 46 at a time point soon after the fistula is created may minimize the risk of body fluid leakage into the tissue surrounding the stomach 62 and biliary duct 68, in addition to minimizing trauma and damage to the tissue surrounding the stomach 62 and biliary duct 68. Further, engagement of the tip member 28 to the wall of the biliary duct 68 prior to the deployment of the stent 46 may stabilize the stent delivery system 10 during deployment of the stent 46, thereby permitting more precise deployment of the stent 46. The precise deployment of the stent 46 may minimize the risk of body fluid leakage into the tissue surrounding the stomach 62 and biliary duct 68, in addition to minimizing trauma and damage to the tissue surrounding the stomach 62 and biliary duct 68.

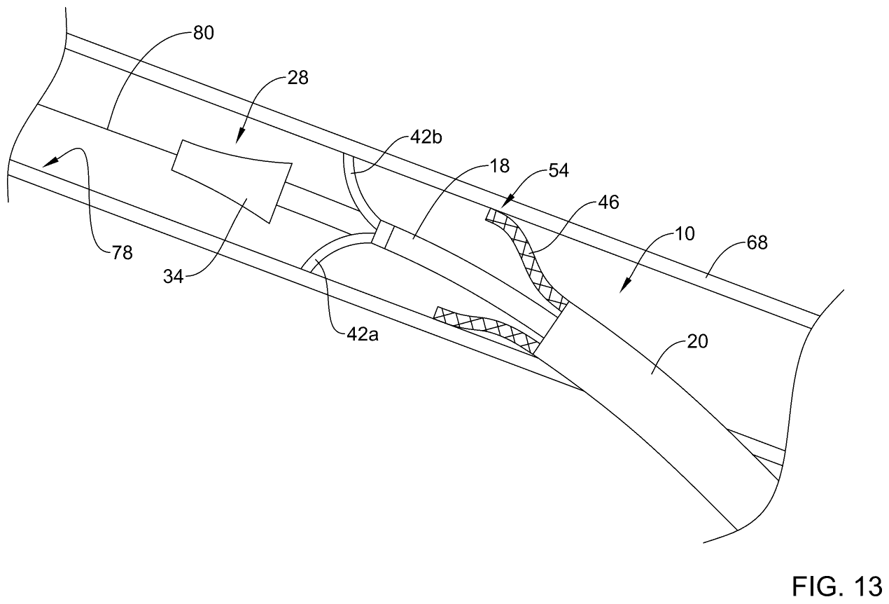

FIG. 11 illustrates the distal end region of the system 10 tracked over the guidewire 80 to a position in which the tip member 28 of the system 10 is positioned within the lumen of the biliary duct 68. Further, it can be appreciated that FIG. 11 illustrates a configuration in which the distal end region 54 of the stent 46 (shown in FIG. 13) is positioned in the lumen of the biliary duct 68 and the proximal end region 56 of the stent 46 (shown in FIG. 15) is positioned in the gastric lumen of the stomach 62. It can be appreciated from FIG. 11 that the engagement members 42a, 42b, 42c, 42d (shown in FIG. 3) and the stent 46 (shown in FIG. 8) are covered by the outer shaft 20.

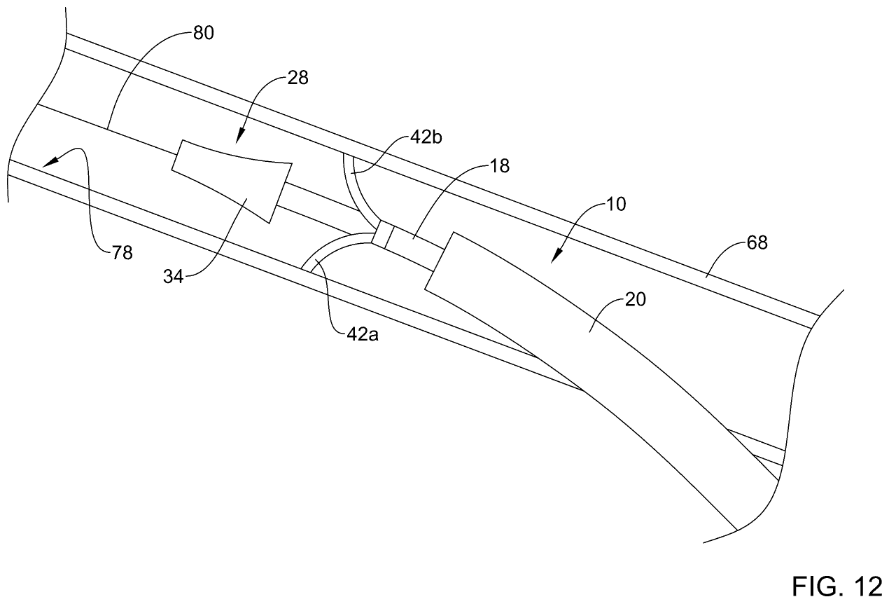

FIG. 12 illustrates the distal-to-proximal retraction of the outer shaft 20 relative to the tip member 28, the engagement members 42a, 42b and the stent 46. As described herein with respect to FIG. 1, the distal-to-proximal retraction of the outer shaft 20 may be accomplished via a user grasping the handle 12 (shown in FIG. 1) with one hand while using their opposite hand to grasp and pull the grip member 26 (shown in FIG. 1) toward the handle 12, thereby translating the outer shaft 20 relative to the tip member 28, the engagement members 42a, 42b, the stent 46 and the inner shaft 18. FIG. 12 further illustrates that the distal-to-proximal retraction of the outer shaft 20 relative to the inner shaft 18 uncovers the engagement members 42a, 42b, 42c, 42d (it can be appreciated that the engagement members 42c, 42d are hidden from view in FIG. 12). As described herein, FIG. 12 illustrates that uncovering the engagement members 42a, 42b, 42c, 42d may permit the engagement members 42a, 42b, 42c, 42d to expand (e.g., flex) radially outward from a first constrained configuration to a second unconstrained configuration, whereby each of the engagement members 42a, 42b, 42c, 42d temporarily engage the inner surface 78 of the biliary duct 68 prior to the deployment of the stent 46 (shown in FIG. 13).

FIG. 13 illustrates further retraction of the outer shaft 20 relative to the tip member 28, the engagement members 42a, 42b, the stent 46 and the inner shaft 18. FIG. 13 illustrates the outer shaft 20 being retracted in a distal-to-proximal direction while the engagement members 42a, 42b, 42c, 42d are temporarily engaged and anchored to the inner surface 78 of the biliary duct 68. The distal-to-proximal retraction of the outer shaft 20 uncovers the stent 46, thereby permitting the stent 46 to shift from a constrained configuration to an expanded configuration. It can be appreciated that the distal-to-proximal retraction of the outer shaft 20 may progressively uncover the stent 46 from distal end region 54 to the proximal end region 56 (shown in FIG. 15). FIG. 13 illustrates the distal end region 54 of the stent 46 expanded within the lumen of the biliary duct 68. As discussed herein, engagement of the engagement members 42a, 42b, 42c, 42d with the inner surface 78 of the biliary duct 68 while the stent 46 is deployed may minimize the movement of the stent delivery system 10 and/or the endoscope during deployment, thereby improving the precision of the placement of the stent 46 within the a target body lumen (e.g., biliary duct, hepatic duct, bile duct, etc.) of a patient.

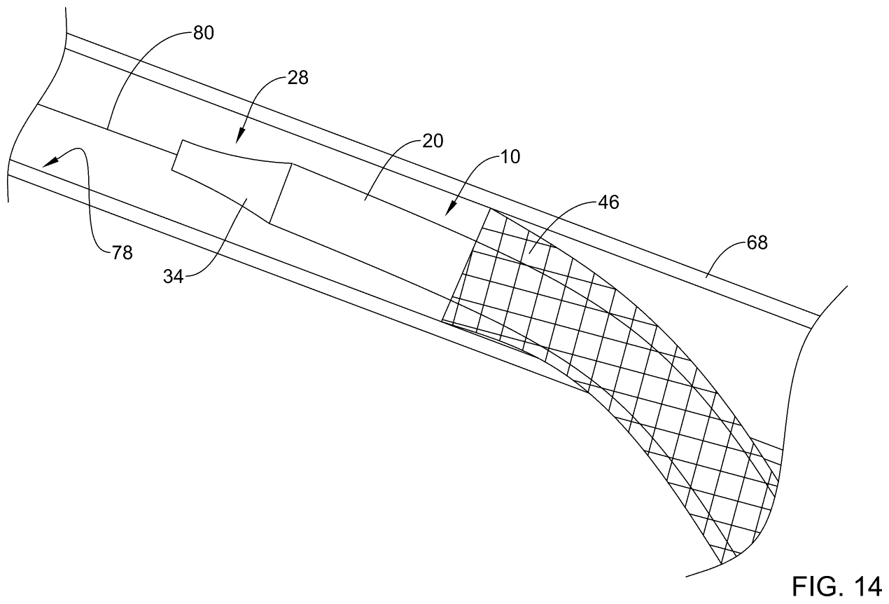

FIGS. 14-15 illustrate that after the outer shaft 20 has been retracted in a proximal-to-distal direction to a position in which the entire length of the stent 46 is deployed (e.g., FIG. 15 illustrates a position in which the distal end region 54 of the stent 46 is deployed in a lumen of the biliary duct 68 and the proximal end region 56 of the stent 46 is deployed in the gastric lumen of the stomach 62), the outer shaft 20 may be advanced in a proximal-to-distal direction through the lumen of the stent 46, whereby the advancement of the outer shaft 20 pulls the engagement members 42a, 42b, 42c, 42d away from their engagement with the inner surface 78 of the biliary duct 68. It can be appreciated that the continued advancement of the outer shaft 20 may position the outer shaft 20 adjacent to the tapered portion 34 of the tip member 28, thereby positioning the outer shaft 20 over top of the engagement members 42a, 42b, 42c, 42d. It can be appreciated that the proximal-to-distal advancement of the outer shaft 20 over top of the engagement members 42a, 42b, 42c, 42d may re-position the engagement members 42a, 42b, 42c, 42d within the channels (e.g., channels 44a, 44b shown in FIG. 3 and channel 44d shown in FIG. 9).

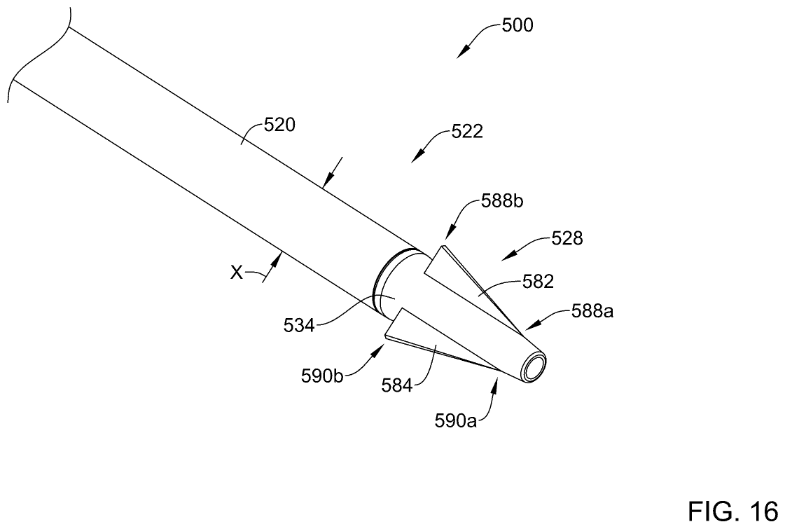

FIG. 16 illustrates a perspective view of the distal end region of another example system 500 (e.g., stent delivery system). The system 500 may be similar in form and function to the system 10 described herein. FIG. 16 illustrates a distal end region 522 of an outer shaft 520 positioned adjacent to a tip member 528. The outer shaft 20 may have an outer diameter X. In some examples, the outer diameter X may be about 2.0 mm (0.079 inches) to about 4.0 mm (0.157 inches). It can be appreciated from FIG. 16 that the distal end of the outer shaft 520 may engage (e.g., abut, contact, etc.) a proximal facing surface of the tip member 528. FIG. 16 further illustrates that the tip member 528 may include distal tapered portion 534.

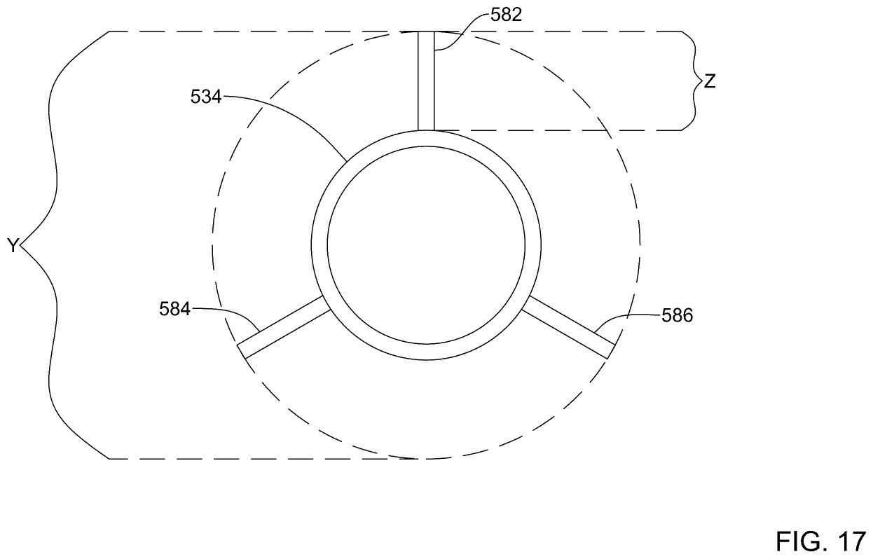

FIGS. 16-17 further illustrates that the tip member 528 may further include one or more fins 582, 584, 586 (shown in FIG. 17) attached to the outer surface of the tapered portion 534. It can be appreciated that the fins 582, 584, 586 may extend radially away from the outer surface of the tapered portion 534. Additionally, FIG. 16 further illustrates that the fin 582 may taper along the longitudinal axis of the tapered portion 534. For example, the fin 582 may taper longitudinally from the distal end 588a of the fin 582 to the proximal end 588b of the fin 582. Similarly, FIG. 16 illustrates that the fin 584 may taper along the longitudinal axis of the tapered portion 534. For example, the fin 584 may taper longitudinally from the distal end 590a of the fin 584 to the proximal end 590b of the fin 584. It can be appreciated that the fin 586 (shown in FIG. 17) may taper similarly to the fins 582, 584 but is hidden from view in FIG. 16.

FIG. 17 illustrates that the tip member 528 including three fins 582, 584, 586 positioned along the outer surface of the tapered portion 534 of the tip member 528. However, it can be appreciated that the tip member 528 may include 1, 2, 3, 4, 5, 6, 7, 8 or more fins circumferentially spaced about the outer surface of the tapered portion 534. Further, in some examples, the fins 582, 584, 586 may be spaced substantially equidistant from one another around the circumference of the tapered portion 534 of the tip member 528. However, in other examples, the fins 582, 584, 586 may be spaced substantially unequally around the circumference of the of the tapered portion 534 of the tip member 528.

FIG. 17 illustrates that the proximal ends of each of the fins 582, 584, 586 may extend radially away from the outer surface of the tapered portion 534 a length Z. In some examples, the maximum length Z may be about 2.5 mm (0.098 inches) to about 4.5 mm (0.177 inches). Accordingly, it can be appreciated that the outer extent of each of the fins 582, 584, 586 may define an outer fin diameter Y. In some examples, the outer fin diameter Y may be about 6.0 mm (0.236 inches) to about 14.0 mm (0.472 inches). Further, in some examples, the ratio of the outer fin diameter Y to the outer diameter X of the outer shaft 20 may be about 3:2 to about 7:1, or about 5:1, or about 7:2, or about 3:1, or about 2:1, or about 3:2.

It can be appreciated that, when positioned in a body vessel (e.g., biliary duct, hepatic duct, bile duct, etc.), the fins 582, 584, 586 may be configured to temporarily engage a wall of a target body lumen (e.g., biliary duct, hepatic duct, bile duct, etc.) while a stent (such as stent 46 shown in FIG. 8) is deployed from the stent delivery system 10. Engagement of the fins 582, 584, 586 with the inner surface of a target body lumen (e.g., biliary duct, hepatic duct, bile duct, etc.) while a stent is deployed may minimize the movement of the stent delivery system 10 and/or the endoscope during deployment, thereby improving the precision of the placement of a covered drainage stent within the a target body lumen (e.g., biliary duct, hepatic duct, bile duct, etc.) of a patient. In some examples, the fins 582, 584, 586 may be configured to collapse and/or fold along the tapered portion 534 of the tip member 528.

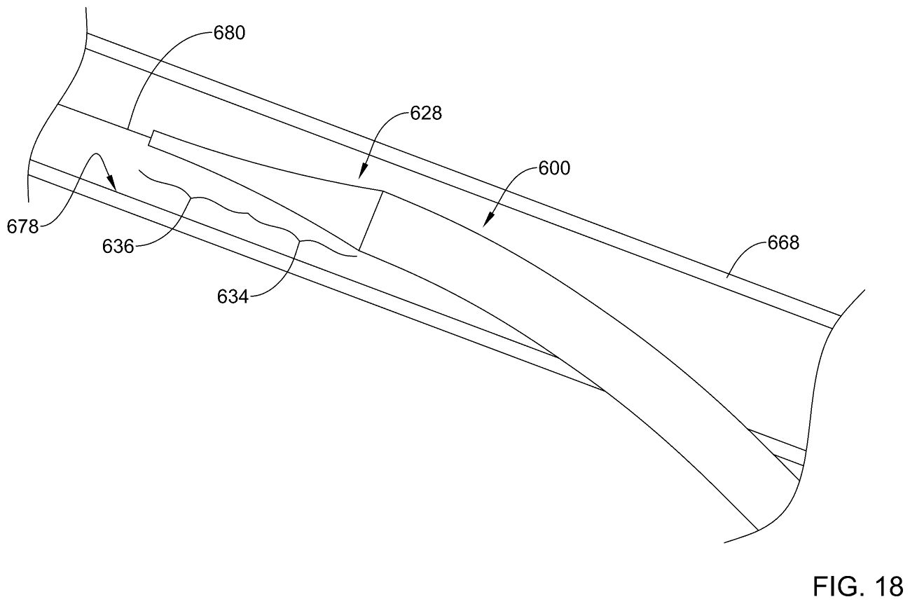

FIG. 18 illustrates a perspective view of the distal end region of another example system 600 (e.g., stent delivery system). The system 600 may be similar in form and function the system 10 described herein. FIG. 18 illustrates that the tip member 628 may include a distal portion 636 and a tapered proximal portion 634. FIG. 18 further illustrates the tip member 628 may include a lumen within which a guidewire 680 may extend. Accordingly, FIG. 18 illustrate the tip member 628 tracked over a guidewire 680 to a position in which the tip member 628 of the system 600 is positioned within the lumen of a body vessel 668 (e.g., biliary duct, hepatic duct, bile duct, etc.).

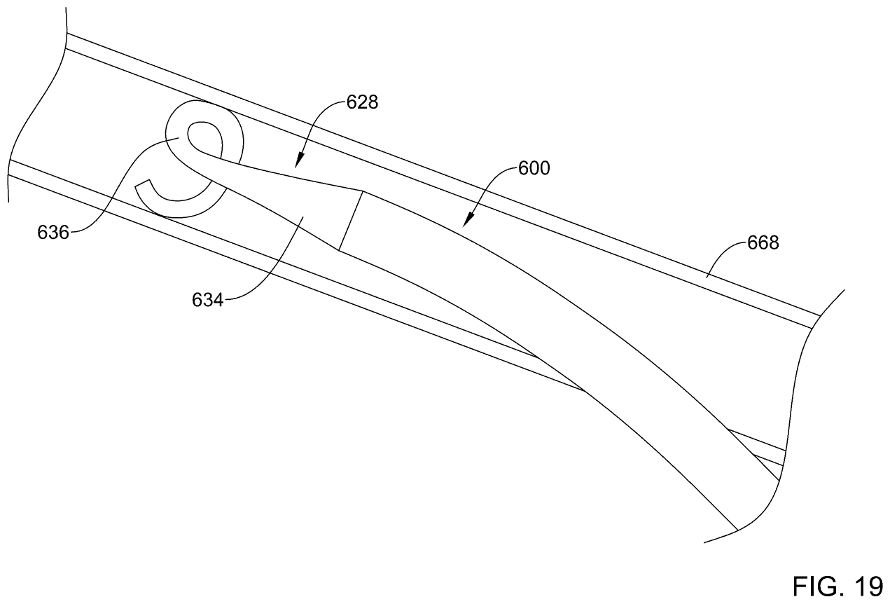

FIGS. 18-19 further illustrate that the distal portion 636 of the tip member 628 may be configured to shift between a first configuration (shown in FIG. 18) whereby the distal portion 636 assumes the substantially straight shape of the guidewire 680 extending through the distal portion 636 and a second configuration (shown in FIG. 19) whereby the distal portion 636 assumes a substantially curved (e.g., pigtail shape, spiral shape, etc.) configuration when the guidewire 680 is retracted and removed from the distal portion 636.

It can be appreciated that the distal portion 636 may be constructed with a pre-set shape such as the curved shape shown in FIG. 19. The tip member 628 may be constructed such that the distal portion 636 is biased to form the substantially curved shape shown in FIG. 19 when a guidewire is not extending within the lumen of the tip member 628. In other words, the tip member 628 may be constructed from a material which permits the distal portion 363 to be constructed with a pre-set shape (such as the curved shape shown in FIG. 19), yet also be flexible enough to be shift from a pre-set curved configuration to a substantially straight configuration when a guidewire 680 passes through the lumen of the tip member 628. A non-exhaustive list of example materials which may be used to construct the tip 628 are disclosed herein.

Further, it can be appreciated that the substantially curved (e.g., pigtail shape, spiral shape, etc.) of the distal portion 636 may be configured to temporarily engage the inner surface 678 of a target body lumen (e.g., biliary duct 668) while a stent (such as stent 46 shown in FIG. 8) is deployed from the stent delivery system 600. Engagement of the distal portion 636 with the inner surface of a target body lumen (e.g., biliary duct, hepatic duct, bile duct, etc.) while a stent is deployed may minimize the movement of the stent delivery system 600 and/or the endoscope during deployment, thereby improving the precision of the placement of a covered drainage stent within the a target body lumen (e.g., biliary duct, hepatic duct, bile duct, etc.) of a patient.

Accordingly, it can be appreciated that after the system 600 is tracked over a guidewire 680 to a position in which the tip member 628 of the system 600 is positioned within the lumen of the biliary duct 668 (as shown in FIG. 18), the guidewire 680 may be retracted and removed from the lumen of the tip member 628, thereby permitting the distal portion 636 of the tip member 628 to shift from a substantially straight configuration (shown in FIG. 18) to the substantially pre-set, curved (e.g., pigtail shape, spiral shape, etc.) configuration (shown in FIG. 19) which temporarily engages the inner surface 678 of the biliary duct 668 during deployment of a stent (such as stent 46 shown in FIG. 8). It can be appreciated that after deployment of a stent (such as stent 46 shown in FIG. 8), the guidewire 680 may be advanced within the lumen of the distal portion 636 of the tip member 628, thereby disengaging the distal portion 636 from the inner surface 678 of the biliary duct 668 and permitting the system 600 (including the tip member 628) to be withdrawn from the patient.

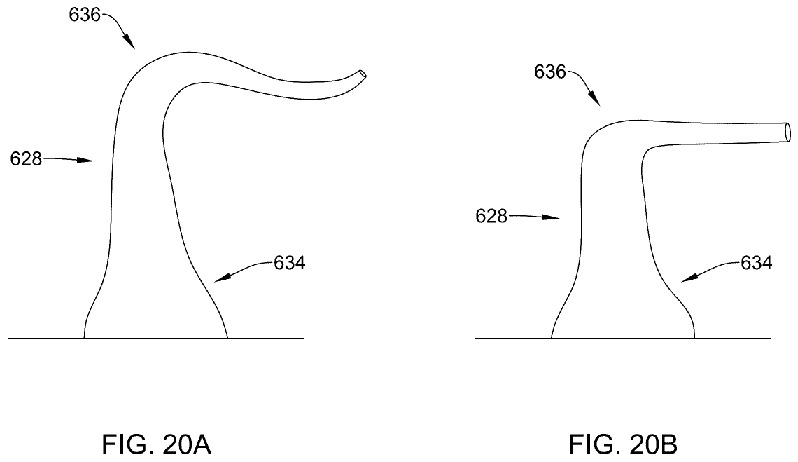

FIGS. 20A-20B schematically depict various example arrangements of the distal portion 636 of the tip member 628. These examples are in addition to the example shape of the distal portion 636 of the tip member 628 shown in FIGS. 18-19. The example shapes of the distal portion 636 of the tip member 628 shown in FIGS. 20A-20B are schematic representations that depict alternative arrangements. The shapes shown in FIGS. 20A-20B are intended as variations of the shape of the distal portion 636 of the tip member 628 shown in FIGS. 18-19, and can be utilized in the system 600 disclosed as alternatives of the distal portion 636 of the tip member 628.

Additionally, it can be appreciated that while the systems (e.g., system 10 and/or any variation thereof) are described herein as “over-the-wire” delivery systems, any of the systems and configurations thereof described herein may be configured as a “rapid exchange” (e.g., single operator exchange) system.

The materials that can be used for the various components of the system 10 or any variation thereof may include those commonly associated with medical devices. For simplicity purposes, the following discussion refers to the various components of the system 10 and/or any variation thereof.

The various components of the system 10 or any variation thereof may be made from or otherwise includes a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A system for positioning a stent along a tissue pathway, the system comprising:
a handle;
an inner shaft having a lumen, a proximal end coupled to the handle and a distal end coupled to a tip member;
an outer shaft having a proximal end region and a lumen, wherein at least a portion of the inner shaft extends within the lumen of the outer shaft; and
a stent positioned between an inner surface of the outer shaft and an outer surface of the inner shaft;
wherein the tip member includes a first engagement member configured to engage and anchor the tip member to a tissue of the tissue pathway before the stent engages the tissue pathway.

2. The system of claim 1, wherein the first engagement member includes a proximal end region and a distal end region, and wherein the proximal end region is coupled to the tip member, and wherein the distal end region is configured to engage and anchor the tip member to a tissue of the tissue pathway.

3. The system of claim 1, wherein the first engagement member is configured to shift between a first constrained configuration and a second expanded configuration.

4. The system of claim 3, wherein the first engagement member is biased to the second expanded configuration.

5. The system of claim 4, wherein the outer shaft is configured to shift between a first position and a second position, and wherein the outer shaft is configured to extend over and maintain the first engagement member in the first constrained configuration when the outer shaft is in the first position.

6. The system of claim 5, wherein shifting the outer shaft from the first position to the second position uncovers the first engagement member, and wherein the first engagement member is configured to shift from the first constrained configuration to the second expanded configuration as the outer shaft uncovers the first engagement member.

7. The system of claim 6, wherein shifting the outer shaft from the second position to the first position covers the first engagement member, and wherein the first engagement member is configured to shift from the second expanded configuration to the first constrained configuration as the outer shaft covers the first engagement member.

8. The system of claim 7, wherein the first engagement member disengages from the tissue of the tissue pathway as the outer shaft is shifted from the second position to the first position and covers the first engagement member.

9. The system of claim 1, wherein the tip member includes a proximal region and a distal region, and wherein the first engagement member is positioned along the proximal region, and wherein the distal region includes a taper.

10. The system of claim 9, wherein the tip member further includes a channel extending along a longitudinal axis of the proximal region, and wherein the engagement member is configured to nest within the channel when in the first constrained configuration.

11. The system of claim 1, wherein the tip member further includes a second engagement member configured to engage and anchor the tip member to a tissue of the tissue pathway before the stent engages the tissue pathway.

12. The system of claim 11, wherein the first engagement member is circumferentially spaced away from the second engagement member along a proximal region of the tip member.

13. The system of claim 1, wherein the first engagement member includes a fin positioned along an outer surface of the tip member.

14. The system of claim 1, wherein the first engagement member is configured to assume a curved configuration to engage and anchor the tip member to the tissue of the tissue pathway.

15. A system for positioning a stent along a tissue pathway, the system comprising:
a handle;
an inner shaft having a lumen, a proximal end coupled to the handle and a distal end coupled to a tip member;
an outer shaft having a proximal end region and a lumen, wherein at least a portion of the inner shaft extends within the lumen of the outer shaft;
a grip member coupled to the proximal end region of the outer shaft;
a stent positioned between an inner surface of the outer shaft and an outer surface of the inner shaft; and
a plurality of engagement members coupled to the tip member;
wherein the plurality of engagement members are configured to engage and anchor the tip member to a tissue of the tissue pathway before the stent engages the tissue pathway.

16. The system of claim 15, wherein each of the plurality of engagement members includes a proximal end region and a distal end region, and wherein the proximal end region of each of the plurality of engagement members is coupled to the tip member, and wherein the distal end region of each of the plurality of engagement members is configured to engage and anchor the tip member to a tissue of the tissue pathway.

17. The system of claim 15, wherein the plurality of engagement members are configured to shift between a first constrained configuration and a second expanded configuration.

18. The system of claim 17, wherein the outer shaft is configured to shift between a first position and a second position, and wherein the outer shaft is configured to extend over and maintain the plurality of engagement members in the first constrained configuration when the outer shaft is in the first position.

19. The system of claim 18, wherein shifting the outer shaft from the first position to the second position uncovers the plurality of engagement members, and wherein plurality of first engagement members are configured to shift from the first constrained configuration to the second expanded configuration as the outer shaft uncovers the plurality of engagement members.

20. A method for positioning a drainage stent between a biliary duct and the stomach, the method comprising:
positioning a distal end region of a stent delivery system within the biliary duct and a proximal end region of the stent delivery system within the stomach, the stent delivery system including:
a handle;
an inner shaft having a lumen, a proximal end coupled to the handle and a distal end coupled to a tip member;
an outer shaft having a proximal end region and a lumen, wherein at least a portion of the inner shaft extends within the lumen of the outer shaft;
a stent positioned between an inner surface of the outer shaft and an outer surface of the inner shaft; and
a plurality of engagement members coupled to the tip member;
retracting the outer shaft in a proximal direction;
engaging the plurality of engagement members to a tissue of the biliary duct;
engaging a distal end of the stent to a tissue of the biliary duct;
engaging a proximal end of the stent to a tissue of the stomach;
advancing the outer shaft in a distal direction; and
disengaging the plurality of engagement members to a tissue of the biliary duct.
